(19) United States
(12) Patent Application Publication
(10) Pub. No.: US 2008/0147049 A1
(43) Pub. Date: Jun. 19, 2008

(54) Devices and methods for catheterization

(76) Inventors: Jamie Glen House, Colorado Springs, CO (US); Kilyn M. Roth, Colorado Springs, CO (US)

Correspondence Address: MOAZZAM & ASSOCIATES, LLC, 7601 LEWINSVILLE ROAD, SUITE 304, MCLEAN, VA, 22102, US

(21) Appl. No.: 11/638,429

(22) Filed: Dec. 14, 2006

(51) Int. Cl. A61M 27/00

(52) U.S. Cl. 604/544

(57) ABSTRACT

Devices and methods are disclosed for a catheterization process, particularly useful for self-performed catheterizations. A catheter is enclosed in a sheath partially attached to the end of the catheter. This leaves an unsealed area at that end leaving the space between the catheter and the sheath in fluid communication with the outside atmosphere. This helps to relieve any pressure built up in that space during use.

### BACKGROUND OF THE INVENTION

1. Field of the Invention

The present invention relates generally to catheter devices and more particularly to urinary catheters having protective sheaths.

2. Background of the Invention

It has become relatively commonplace for the occasional, intermittent or periodic catheterization of an individual's urinary bladder to be employed, as opposed to placement and maintenance of an indwelling catheter that continuously drains urine from the bladder. Short-term or repeated catheterization is appropriate, or even required, for many persons who are in a hospital setting, a nursing home, doctor's office, rehabilitation facility, or in the home. For example, a patient is sometimes catheterized to treat such conditions as urinary retention, the inability to evacuate urine, or for the purpose of obtaining a sterile urine specimen from a patient in a doctor's office or hospital.

The need for intermittent catheterization of an individual sometimes arises due to problems typically associated with long-term use of indwelling catheters, such as infections, urethral damage, and bladder damage. Long-term use of an indwelling catheter is also a risk factor for bladder cancer. This is often the case for persons having a neurogenic urinary condition, such as in a spinal cord injury, multiple sclerosis, stroke, trauma, or other brain injury. Conditions that interfere with the individual's ability to voluntarily void the bladder may also arise post-surgically or as a result of benign prostatic hypertrophy or diabetes. Many of the affected individuals are capable of, and would prefer to perform self-catheterization. For many, the level of risk and discomfort of repeated catheterizations carried out over the course of a day (at 3-6 hour intervals, for example) are offset by the accompanying convenience, privacy, or self-reliance that is achieved. Some of the major difficulties arising in self-catheterization are the lack of satisfactory catheterization kits, the problem of maintaining the required level of sterility during the procedure, and the difficulty of sometimes performing the procedure under conditions of restricted space and privacy.

In assisted, or non self-catheterizations, it is common practice in hospitals to employ a catheterization tray, which typically includes a sterile drape, gloves, a conventional catheter, antiseptic solution, swabs, lubricant, forceps, underpad, and a urine collection container. Assisted catheterization is usually performed with the patient in a supine position. Maintaining a sterile field during the procedure can still be a problem, however, and the “cath tray” procedure is impractical for use with some individuals and situations today.

Many individuals with spinal cord injuries or other neurological diseases routinely perform intermittent catheterization several times a day using conventional catheters or kits and “clean technique.” Clean technique means that the urethral area is cleansed without sterile technique, and efforts are made to avoid contamination of the catheter during the procedure. The user's hands and catheter are not sterile and a sterile field is not maintained. Clean technique is used instead of sterile technique, generally, for two reasons. First, it is very difficult, if not impossible, for individuals who are performing self-catheterization to adhere strictly to sterile technique. Secondly, these individuals are required to catheterize themselves between 3 and 6 times a day, and the cost of a new sterile catheter and the accessories required to perform sterile catheterization becomes excessively expensive for some users. Sometimes an individual will reuse a “cleaned” catheter. As a result, the use of “clean technique” will many times result in contamination and subsequent infection of the urinary tract, causing significant morbidity and cost to the patient and society.

To maintain sterility many catheters are surrounded by sheaths. The user holds the sheath while pulling the catheter through, avoiding direct contact with the catheter before and during insertion. While the user is pulling the catheter through the sheath, the sheath can tend to bunch up at the proximal end. Any excess air is then forced to the distal end. During insertion air builds up throughout the sheath, inflating the sheath to a maximum, interfering or preventing the insertion process. Completion can be impossible to those with limited manual dexterity.

### SUMMARY OF THE INVENTION

The present invention solves many problems associated with conventional catheters, including the problem of pressure build-up, with a technique of partially attaching the sheath to the catheter. The catheter sheath, which surrounds the catheter from the proximal end to the distal end, is secured to only a portion of the circumference of the catheter. The rest of the circumference is loosely attached, allowing excess air pressure to vent to the outside during use. This will allow even users with compromised manual dexterity to complete the self-catheterization process easily and efficiently.

Furthermore, the present invention can include components that further ease the catheterization process. Particular exemplary components include lubrication so that the catheter slides smoothly down the urinary tract, and a guiding tip, which may also have a lubricant reservoir, to give the user something solid to line up the catheter to the urethra. Also, a hydrophilic coating is used on the catheter of certain embodiments to hold the lubricant onto the catheter while in the urinary tract.

An exemplary embodiment of the present invention is a catheter surrounded by a sheath that is partially attached at the distal end. The sheath is heat-sealed around a quarter to a half of the circumference of the catheter. The rest of the circumference is either loosely attached or not at all, allowing air to vent through this opening and prevent a pressure build-up. The sheath could also be partially attached at the proximal end although it is not as ideal.

Another exemplary embodiment of the present invention is a catheter surrounded by a sheath that is attached with a clamp at the distal end. This is a circular clamp about the same circumference as the catheter. The clamp will surround over half of the circumference of the catheter, but not the entirety, leaving an unclamped section to vent air build-up. If necessary the clamp can be removed for more ventilation. The sheath could also be partially attached at the proximal end although it is not as ideal.

Yet another exemplary embodiment of the present invention is a catheter surrounded by a sheath that is partially attached at the distal end and clamped. The sheath is heat-sealed around a quarter to a half of the circumference of the catheter, leaving the rest unattached. A circular clamp is then placed around the portion of the circumference that is unattached, the combination sealing the sheath to the catheter completely. This maintains a near perfect seal, but the clamp must be removed before or during use in order to create a vent allowing built-up air to escape. In this embodiment the clamp must be easy to remove to accommodate those with compromised manual dexterity. The sheath could also be partially attached at the proximal end although it is not as ideal.

### BRIEF DESCRIPTION OF THE DRAWINGS

FIG. 1A shows an external perspective view of a catheter surrounded by a sheath according to an exemplary embodiment of a conventional assembly.

FIG. 1B shows an external perspective view of a catheter surrounded by a sheath with the sheath pulled back to expose the catheter and showing an air pocket created.

FIGS. 2A-C show a cross-sectional view of a catheter with a sheath partially attached to the circumference of the catheter according to an exemplary embodiment of the present invention.

FIG. 2D shows a perspective view of a catheter with a sheath partially clamped to the circumference of the catheter according to an exemplary embodiment of the present invention.

FIGS. 3A-B show a cross-sectional view of a catheter with a sheath clamped to the circumference of the catheter according to an exemplary embodiment of the present invention.

FIG. 4 shows a cross-sectional view of a catheter with a sheath partially attached to the circumference; a clamp seals the rest of the circumference according to an exemplary embodiment of the present invention.

### DETAILED DESCRIPTION OF THE INVENTION

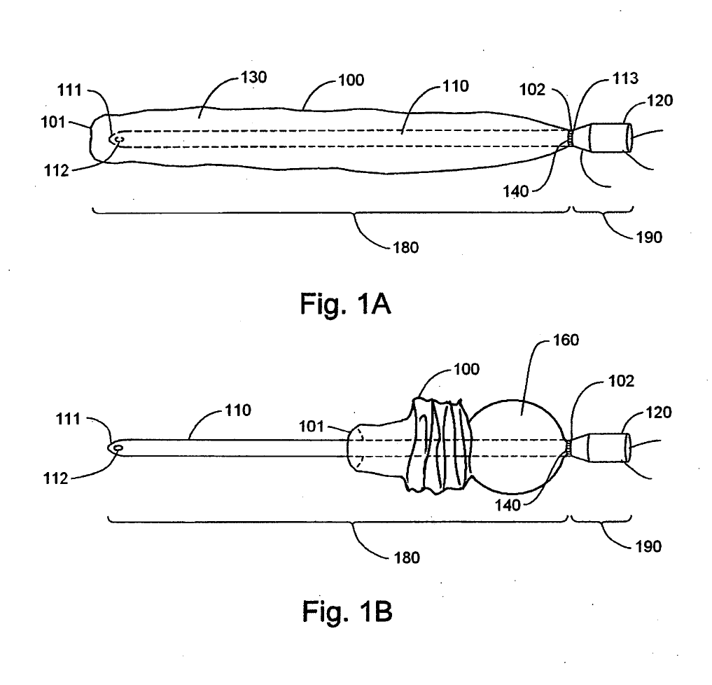

The present invention includes devices and methods for urinary catheterization for patients who want to self-catheterize in a sterile, safe, and efficient manner. Also, the present invention can be used effectively by nurses and other health care professionals in hospitals and clinics. In order to achieve the level of sterility required to avoid infection, a sheath 100, as shown in FIG. 1, is used to cover the portion of the catheter that is insertable into the urethra 180. To ensure sterility, the sheath must cover the catheter 110 from time of storage until it is fully inserted. The user then pulls the catheter 110 through the sheath 100 without touching the catheter 110 itself. As the user pulls the catheter 110 through the sheath 100, excess air builds up 160 throughout the sheath 102. This excess air 160 needs to be released from inside the sheath 100 to the outside atmosphere. The catheter in the present invention is surrounded by a sheath that is partially attached at one end of the catheter. In most instances the sheath is completely sealed at the other end of the catheter but could be potentially attached at both ends.

A conventional assembly for a catheter with a sheath is shown in FIG. 1A. The proximal end of the sheath 101 surrounds the proximal tip of the catheter 111 and is closed at the end. In order to perform a catheterization using this device, the user should first open the proximal end of the sheath 100, exposing the proximal end of the catheter 111, as shown in FIG. 1B. The user then holds the proximal tip of the catheter 111 with the sheath 100 between the user's hand and the catheter 110 and pulls the sheath 100 with the other hand. As the user pulls the sheath 100, which is attached to the catheter at its distal end 113, the catheter 110 will be pushed through the sheath 100 and into the urethra, causing the sheath 100 to bunch up at the proximal end 101.

This process continues until the catheter 110 runs all the way through the urethra and into the bladder. Once inside, fluid from the bladder will stream into the catheter through the opening 112 at the proximal tip of the catheter 111. Provided the catheter distal end 113 is lower in altitude than the catheter proximal end 111, fluid will flow through the catheter, out the outlet 120, and into a receptacle. When the bladder has been drained of all fluid the catheter 110 is then pulled out of the urethra by the user, and disposed.

This partial attachment to the end of the catheter can be achieved in a number of ways. In most cases the attachment concerned will be at the distal end of the catheter 113, the end that is furthest from the patient during use. The proximal end of the catheter 111, the end closest to the patient during use, is most often sealed completely, to be opened prior to use. The distal end 113 is only partially sealed around the circumference of the catheter 110, leaving a portion of the circumference unsealed so that the space between the sheath 100 and the catheter 110 is in fluid communication with the outside atmosphere. Alternatively, the proximal end can also be used for partial sealing.

When the user starts to push the sheath 100 down the catheter 110 during insertion, the sheath 100 bunches up at the distal end 102 causing a pocket of air pressure 160 to build inside of the sheath, as shown in FIG. 1B. With a partially attached sheath 100, this pocket 160 will slowly dissipate or possibly never form, depending on the area left unsealed between the sheath 100 and the catheter 110. If an air pocket 160 does form, the speed that it reduces or dissipates relates directly to the portion of the circumference of the catheter 110 remaining unsealed.

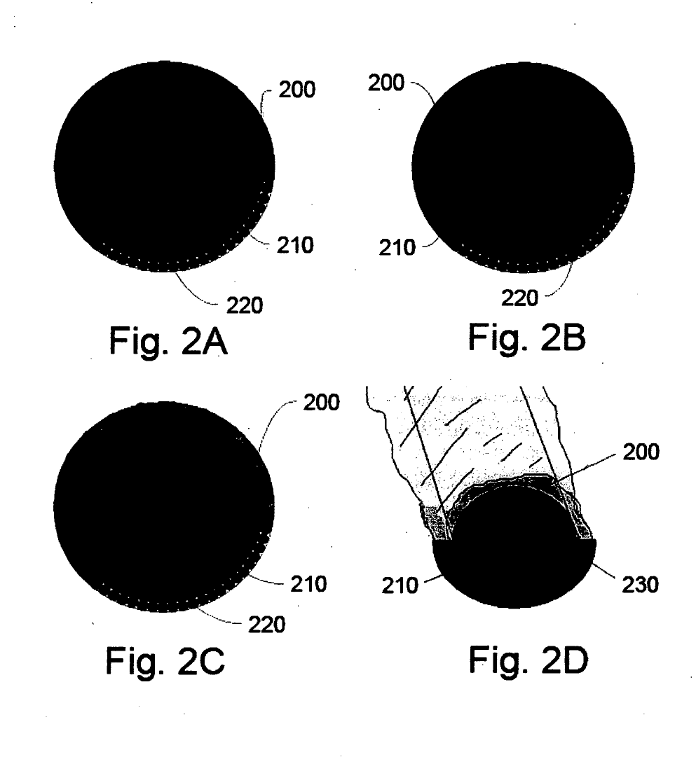

In one exemplary embodiment of the present invention, the sheath 200 is sealed 220 to the end of the catheter 210 about a quarter to a half of the total circumference of the catheter 210 as shown in FIGS. 2A-2C. The sheath 200 can be sealed 220 using a heat sealant, glue, or any other suitable adhesive. This leaves about three quarters to one half of the catheter's 210 circumference unattached. The sheath 200 surrounding this portion can be left like it is as in FIG. 2B, or sealed at a few points around the remainder as in FIG. 2A and 2C. The amount of circumference that is left unsealed has a direct relation to the amount of ventilation and thus the ease of relieving pressure. This should be kept in mind when deciding how much area to seal 220 off initially and how many points, if any, should be sealed 220 throughout the remainder of the circumference.

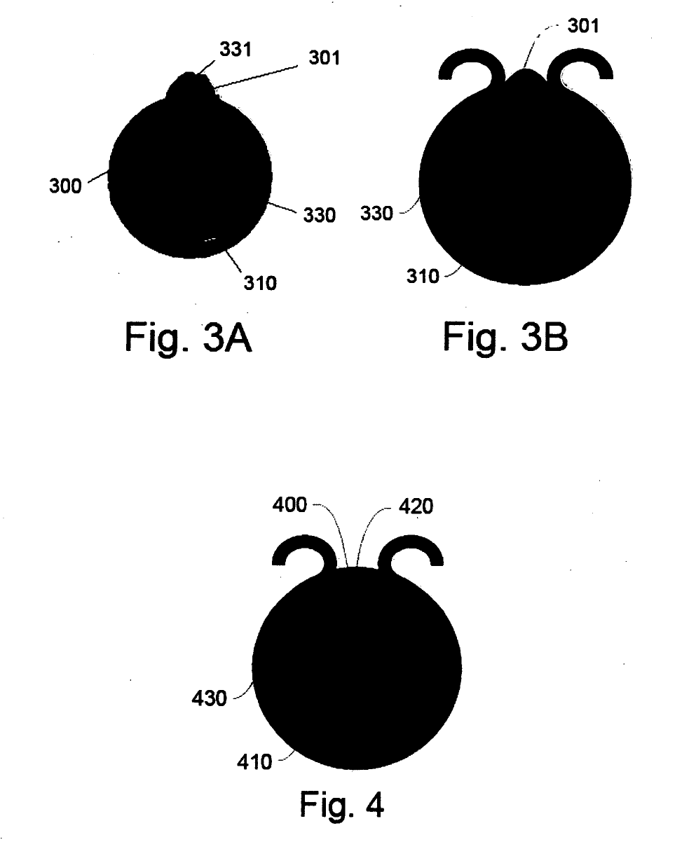

According to another exemplary embodiment of the present invention, a clamp 330 is used to seal the sheath 300 to a portion of the circumference of the catheter 310 as shown in FIGS. 3 and 2D. This clamp 330 can be made from any plastic, ceramic, or like material, possibly even wood. Clamp 310 needs only to be strong enough to remain in a clamped position while attached to the sheath 300 and the catheter 310. The clamp 330 covers a larger portion of the circumference of the catheter 310 than that of the sealed embodiment. The clamp 330 should cover at least half of the circumference, but not completely, allowing a portion of the circumference unsealed 301. FIG. 2D shows a sheath 300 clamped to a catheter 310 around only half of the circumference, while FIG. 3B shows a sheath 300 clamped around almost the entirety of the circumference. FIG. 3A shows a sheath 300 clamped to almost the entirety of the circumference of the catheter, similar to FIG. 3B. However, the ends of the clamp 330 are connected in a fashion not sealing the sheath 300 to the catheter 310, but controlling the area left unsealed 301. The portion of the circumference left unclamped 301 creates a dome 331 which is directly proportional to the amount of ventilation and thus the ease of releasing excess pressure within the sheath 300 during use. FIG. 3A also shows an alternative embodiment wherein the clamp 330 includes a portion 331 that overall can cover the total circumference of the catheter, whether sealed or not sealed.

In yet another exemplary embodiment of the present invention, the sheath 400 is partially sealed 420 to the catheter 410 at one end with a suitable adhesive, then a clamp is used to seal the remainder of the circumference as shown in FIG. 4. The sealed portion 420 can cover about a quarter to a half of the circumference, while the remainder can remain completely unsealed or sealed at only a few points. The clamp 430 most likely overlaps the adhered 420 portion of the sheath 400. This clamp 430 should be removed before use in order to allow pressure to be relieved after the user pulls the sheath 400 down.

It should also be noted that although the sealed portion has been described above as covering about a quarter to a half of the circumference, various other amounts may also be effective. For example, the heat seal could also be one point of attachment as small as it takes to stick (or tack down with adhesion technique). A single dot or point could be enough to secure the sheath to the catheter with minimal attachment.

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. A catheter assembly comprising:
a urinary catheter; and
a sheath substantially surrounding the catheter;
wherein the sheath is partially attached to an end of the catheter allowing excess air to escape.

2. The catheter in claim 1, wherein the sheath is partially sealed to a circumference of the catheter.

3. The catheter in claim 1, wherein the sheath is partially attached to a distal end of the catheter.

4. The catheter in claim 1, wherein the sheath is partially attached to a proximal end of the catheter.

5. The catheter in claim 1, wherein the sheath is partially attached by heat sealant and/or glue.

6. The catheter in claim 1, wherein the sheath is attached between one quarter and one half of the total circumference of the catheter.

7. The catheter in claim 6, wherein the remainder of the circumference is sealed at a plurality of points.

8. The catheter in claim 1, wherein the sheath is attached at a single point in the total circumference of the catheter.

9. The catheter in claim 1, wherein the sheath is attached with a clamp.

10. The catheter in claim 9, wherein the clamp surrounds at least half but not the entirety of the circumference of the catheter.

11. The catheter in claim 10, wherein the clamp is made from plastic, metal and/or wood.

12. The catheter in claim 1, wherein the sheath is partially sealed to the circumference of the catheter then clamped with a removable clamp around the unsealed portion of the circumference.

13. A catheter assembly comprising:
a urinary catheter; and
a sheath substantially surrounding the catheter;
wherein the sheath is partially attached to an end of the catheter to leak excess pressure from an interior of the sheath.

14. The catheter in claim 13, wherein the sheath is partially sealed to a circumference of the catheter.

15. The catheter in claim 13, wherein the sheath is partially attached to a distal end of the catheter.

16. The catheter in claim 13, wherein the sheath is partially attached to a proximal end of the catheter.

17. The catheter in claim 13, wherein the sheath is attached with a clamp.

18. The catheter in claim 17, wherein the clamp surrounds at least half but not the entirety of the circumference of the catheter.

19. The catheter in claim 17, wherein the clamp surrounds the entirety of the circumference of the catheter.

20. The catheter in claim 13, wherein the sheath is partially sealed to the circumference of the catheter, then clamped with a removable clamp around the unsealed portion of the circumference.

21. A method of relieving pressure from inside a sheath of a urinary catheter comprising:
pushing the sheath down the catheter; and
forcing an amount of excess air out of a partial opening between the sheath and the catheter.

22. The method in claim 21, further comprising removing a clamp from the sheath and catheter before use.
